(19) United States
(12) Patent Application Publication
(10) Pub. No.: US 2014/0323876 A1
(43) Pub. Date: Oct. 30, 2014

(54) SYSTEMS AND METHODS FOR DETERMINING FLUID RESPONSIVENESS IN THE PRESENCE OF GAIN CHANGES AND BASELINE CHANGES

(71) Applicant: Covidien LP, Mansfield, MA (US)

(72) Inventors: Scott McGonigle, Edinburgh (GB); Paul S. Addison, Edinburgh (GB); Rui Wang, Livingston (GB); James N. Watson, Edinburgh (GB)

(73) Assignee: Covidien LP, Mansfield, MA (US)

(21) Appl. No.: 14/260,200

(22) Filed: Apr. 23, 2014

Related U.S. Application Data
(60) Provisional application No. 61/815,917, filed on Apr. 25, 2013.

(51) Int. Cl. A61B 5/00

(52) U.S. Cl. CPC A61B 5/0059; A61B 5/7225
USPC 600/476

(57) ABSTRACT

Methods and systems are provided for determining fluid responsiveness based on physiological signals. The system may detect gain changes or excessive baseline modulations. In some embodiments, based on the detected gain changes or excessive baseline modulations, the system may ignore portions of physiological signals and determine a parameter indicative of fluid responsiveness based on a plurality of amplitudes determined from other portions of the physiological signals. In some embodiments, based on the detected gain changes or excessive baseline modulations, the system may determine fluid responsiveness, or refrain from determining fluid responsiveness.

### CROSS REFERENCE TO RELATED APPLICATIONS

This application claims the benefit of U.S. Provisional Application No. 61/815,917, filed Apr. 25, 2013, which is hereby incorporated by reference herein in its entirety.

### SUMMARY

The present disclosure relates to determining fluid responsiveness, and more particularly relates to determining fluid responsiveness in the presence of gain changes and baseline changes.

Methods and systems are provided for determining fluid responsiveness of a subject. In some embodiments, physiological signals are received by a system. The system may detect the presence of gain changes or baseline changes. The system may calculate fluid responsiveness based on the presence or absence of gain changes and/or baseline changes.

The present disclosure provides embodiments for a physiological monitor that monitors fluid responsiveness of a subject. The system comprises a signal generating module, a gain change identification module, and a fluid responsiveness parameter determination module. The signal generating module is configured to generate a physiological signal that is indicative of light attenuated by a subject and the gain change identification module is configured to identify a gain change in the signal generating module. The fluid responsiveness parameter determination module is configured to receive the physiological signal from the signal generating module and receive the gain change identification from the gain change identification module. The fluid responsiveness parameter determination module is further configured to determine a first plurality of amplitudes in a first portion of the physiological signal, ignore a second portion of the physiological signal, subsequent to the first portion, based on the received gain change identification, and determine a second plurality of amplitudes in a third portion of the physiological signal, subsequent to the second portion. The fluid responsiveness parameter determination module is further configured to determine the parameter indicative of fluid responsiveness based on the first plurality of amplitudes and the second plurality of amplitudes, and not based on the ignored second portion of the physiological signal.

The present disclosure provides embodiments for a physiological monitor that monitors fluid responsiveness of a subject. The system comprises an input configured to receive a physiological signal, a baseline gradient detection module configured to detect excessive baseline modulations of the physiological signal that exceed a predetermined threshold, and a fluid responsiveness parameter determination module. The fluid responsiveness parameter determination module is configured to receive the physiological signal, receive information indicative of excessive baseline modulations from the baseline gradient detection module, determine a fluid responsiveness parameter based on the physiological signal, and refrain from determining the fluid responsiveness parameter based on the information indicative of excessive baseline modulations.

The present disclosure provides embodiments for a method of determining fluid responsiveness of a subject comprising receiving a physiological signal, detecting a gain change in the physiological signal, determining a fluid responsiveness parameter based on the physiological signal, and refraining from determining the fluid responsiveness parameter based on the detected gain change in the physiological signal.

The present disclosure provides embodiments for a method of determining fluid responsiveness of a subject comprising receiving a physiological signal, detecting excessive baseline modulations of the physiological signal, determining a fluid responsiveness parameter based on the physiological signal, and refraining from determining the fluid responsiveness parameter based on the detected excessive baseline modulations of the physiological signal.

The present disclosure provides embodiments for a method of determining fluid responsiveness of a subject comprising receiving a physiological signal and receiving a gain change identification for the physiological signal. The method further comprises determining a first plurality of amplitudes in a first portion of the physiological signal, ignoring a second portion of the physiological signal subsequent to the first portion based on the gain change identification, and determining a second plurality of amplitudes in a third portion of the physiological signal. The method further comprises determining fluid responsiveness based on the first plurality of amplitudes and the second plurality of amplitudes, and not based on the ignored second portion of the physiological signal.

The present disclosure provides embodiments for a method of determining fluid responsiveness of a subject comprising receiving a physiological signal and receiving information indicative of excessive baseline modulations of the physiological signal. The method further comprises determining a first plurality of amplitudes in a first portion of the physiological signal, ignoring a second portion of the physiological signal subsequent to the first portion based on the information indicative of excessive baseline modulations, and determining a second plurality of amplitudes in a third portion of the physiological signal. The method further comprises determining fluid responsiveness based on the first plurality of amplitudes and the second plurality of amplitudes, and not based on the ignored second portion of the physiological signal.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal that may be generated by a sensor in accordance with some embodiments of the present disclosure;

FIG. 3 is a perspective view of an illustrative physiological monitoring system in accordance with some embodiments of the present disclosure;

FIG. 4 shows an illustrative plot of a PPG waveform reflecting respiratory modulations in accordance with some embodiments of the present disclosure;

FIG. 5 shows an illustrative diagram of a PPG signal divided into segments in accordance with some embodiments of the present disclosure;

FIG. 6 shows an illustrative plot of a PPG signal during a gain change in accordance with some embodiments of the present disclosure;

FIG. 7 shows an illustrative plot of a PPG signal during excessive baseline modulations in accordance with some embodiments of the present disclosure;

FIG. 8 shows illustrative steps for determining fluid responsiveness in accordance with some embodiments of the present disclosure;

FIG. 9 shows illustrative steps for determining fluid responsiveness in accordance with some embodiments of the present disclosure;

FIG. 10 shows an illustrative physiological monitor for monitoring fluid responsiveness in a subject in accordance with some embodiments of the present disclosure; and

FIG. 11 shows an illustrative physiological monitor for monitoring fluid responsiveness in a subject in accordance with some embodiments of the present disclosure.

### DETAILED DESCRIPTION OF THE FIGURES

The present disclosure is directed towards determining fluid responsiveness of a subject. In particular, a monitor is configured to determine fluid responsiveness in a subject based on physiological signals in the presence gain changes and baseline changes.

Fluids are commonly delivered to a patient in order to improve the patient's hemodynamic status. Fluid is delivered with the expectation that it will increase the patient's cardiac preload, stroke volume, and cardiac output, resulting in improved oxygen delivery to the organs and tissue. Fluid delivery may also be referred to as volume expansion, fluid therapy, fluid challenge, or fluid loading. However, improved hemodynamic status is not always achieved by fluid loading. Moreover, inappropriate fluid loading may worsen a patient's status, such as by causing hypovolemia to persist (potentially leading to inadequate organ perfusion), or by causing hypervolemia (potentially leading to peripheral or pulmonary edema).

Respiratory variation in the arterial blood pressure waveform is known to be a good predictor of a patient's response to fluid loading, or fluid responsiveness. Fluid responsiveness represents a prediction of whether such fluid loading will improve blood flow within the patient. Fluid responsiveness refers to the response of stroke volume or cardiac output to fluid administration. A patient is said to be fluid responsive if fluid loading does accomplish improved blood flow, such as by an improvement in cardiac output or stroke volume index by about 15% or more. In particular, the pulse pressure variation (PPV) parameter from the arterial blood pressure waveform has been shown to be a good predictor of fluid responsiveness. This parameter can be monitored while adding fluid incrementally, until the PPV value indicates that the patient's fluid responsiveness has decreased, and more fluids will not be beneficial to the patient. This treatment can be accomplished without needing to calculate blood volume or cardiac output directly. This approach, providing incremental therapy until a desired target or endpoint is reached, may be referred to as goal-directed therapy (GDT).

However, determining the PPV is an invasive procedure, requiring the placement of an arterial line in order to obtain the arterial blood pressure waveform. This invasive procedure is time-consuming and presents a risk of infection to the patient. Respiratory variation in a photoplethysmograph (PPG) signal may provide a non-invasive alternative to PPV. The PPG signal can be obtained non-invasively, such as from a pulse oximeter. One measure of respiratory variation in the PPG is the Delta POP metric, which is a measure of the strength of respiratory-induced amplitude modulations of the PPG. This metric assesses changes in the pulse oximetry plethysmograph, and is abbreviated as ΔTOP or DPOP. Typically, DPOP is determined instantaneously over a first window, which may be a fixed period or may be a period corresponding to a breath of the subject, and the DPOP value used for diagnosis is an average of the instantaneous DPOP values determined for multiple windows. Thus it is desirable to ensure that the instantaneous DPOP values used in averaging are of good quality.

In accordance with the present disclosure, events that may introduce error to the instantaneous DPOP values are detected, and corresponding portions of the signal are excluded from the calculation of DPOP. For example, a monitor may detect time periods corresponding to a gain change and refrain from calculating DPOP during those time periods, or exclude instantaneous DPOP values calculated during those time periods from an average DPOP. Similarly, a monitor may detect time periods corresponding to excessive baseline modulations in the PPG signal, and refrain from calculating DPOP during those time periods, or exclude instantaneous DPOP values calculated during those time periods from an average DPOP.

The foregoing techniques may be implemented in an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation invasively by analyzing a blood sample taken from the patient). Pulse oximeters may be included in patient monitoring systems that measure and display various blood flow characteristics including, but not limited to, the blood oxygen saturation (e.g., arterial, venous, or both). Such patient monitoring systems, in accordance with the present disclosure, may also measure and display additional or alternative physiological parameters such as pulse rate, respiration rate, respiration effort, blood pressure, hemoglobin concentration (e.g., oxygenated, deoxygenated, and/or total), systemic vascular resistance, mean arterial pressure, cardiac output, central venous pressure, oxygen demand, adaptive filter parameters, fluid responsiveness parameters, any other suitable physiological parameters, or any combination thereof.

Pulse oximetry may be implemented using a photoplethysmograph. Pulse oximeters and other photoplethysmograph devices may also be used to determine other physiological parameter and information as disclosed in: J. Allen, “Photoplethysmography and its application in clinical physiological measurement,” Physiol. Meas., vol. 28, pp. R1-R39, March 2007; W. B. Murray and P. A. Foster, “The peripheral pulse wave: information overlooked,” J. Clin. Monit., vol. 12, pp. 365-377, September 1996; and K. H. Shelley, “Photoplethysmography: beyond the calculation of arterial oxygen saturation and heart rate,” Anesth. Analg., vol. 105, pp. S31-S36, December 2007; all of which are incorporated by reference herein in their entireties.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, around or in front of the ear, and locations with strong pulsatile arterial flow. Suitable sensors for these locations may include sensors that detect reflected light.

The oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, an inverted signal, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine arterial blood oxygen saturation using two wavelengths of light and a ratio-of-ratios calculation. As another example, the system may determine regional blood oxygen saturation using two wavelengths of light and two detectors located at different distances from the emitters. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to detect ambient signal levels, reduce overlap of the light drive pulses, allow time for light sources to stabilize, allow time for detected light signals to stabilize or settle, reduce heating effects, reduce power consumption, for any other suitable reason, or any combination thereof.

It will be understood that the techniques described herein are not limited to pulse oximeters and may be applied to any suitable physiological monitoring device.

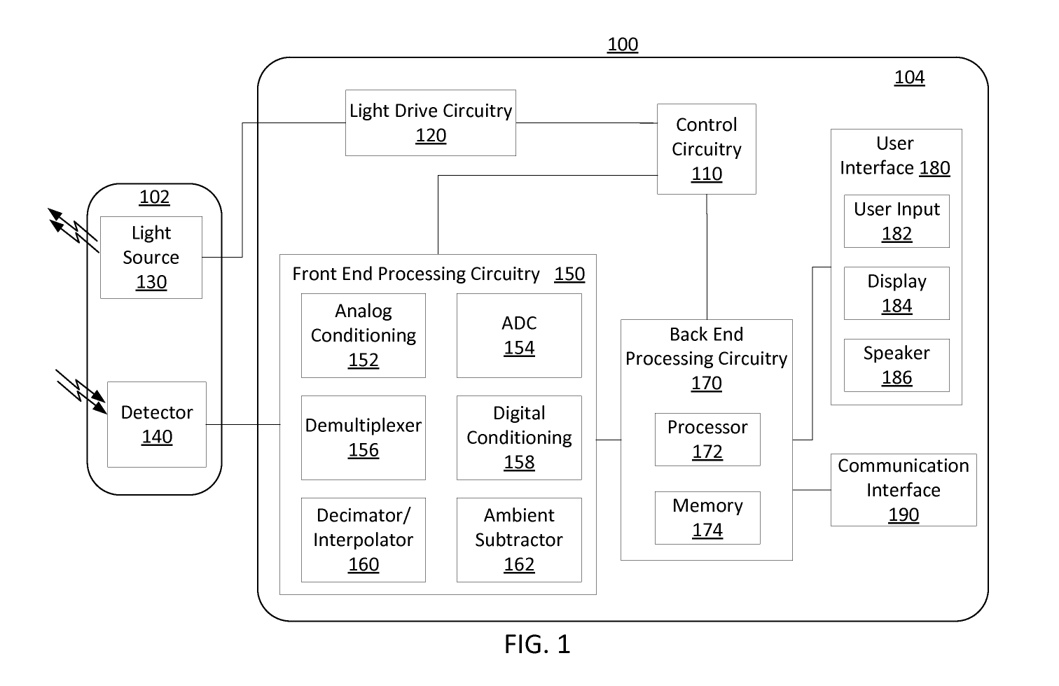

The following description and accompanying FIGS. 1-11 provide additional details and features of some embodiments of the present disclosure.

FIG. 1 shows a block diagram of illustrative physiological monitoring system 100 in accordance with some embodiments of the present disclosure. System 100 may include a sensor 102 and a monitor 104 for generating and processing sensor signals that include physiological information of a subject. In some embodiments, sensor 102 and monitor 104 may be part of an oximeter. In some embodiments, system 100 may include more than one sensor 102.

Sensor 102 of physiological monitoring system 100 may include light source 130 and detector 140. Light source 130 may be configured to emit photonic signals having one or more wavelengths of light (e.g. red and IR) into a subject's tissue. For example, light source 130 may include a red light emitting light source and an IR light emitting light source, e.g. red and IR light emitting diodes (LEDs), for emitting light into the tissue of a subject to generate sensor signals that include physiological information. In one embodiment, the red wavelength may be between about 600 nm and about 750 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104. Although only one detector 140 is depicted in FIG. 1, in some embodiments, sensor 102 may include additional detectors located at different distances from the light source 130. In embodiments with additional detectors, the sensitivity of the additional detectors may vary based on the distance between the detector and light source 130 such that a far detector may be more sensitive to light than a near detector.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

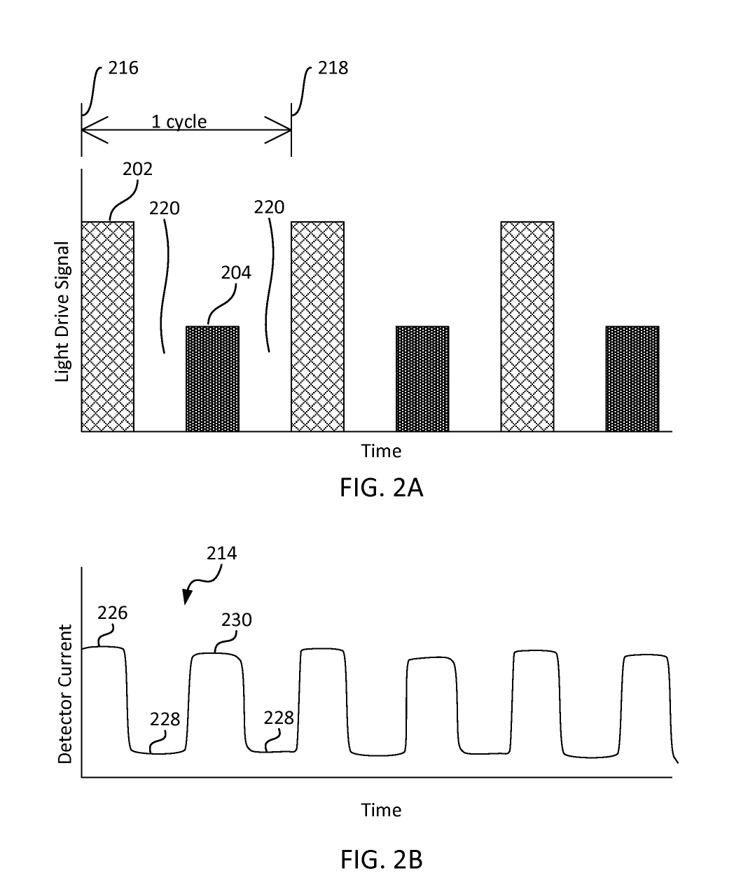

Light drive circuitry 120, as discussed above, may be configured to generate a light drive signal that is provided to light source 130 of sensor 102. The light drive signal may, for example, control the intensity of light source 130 and the timing of when light source 130 is turned on and off. In some embodiments, the intensity of light source 130 may be set based on a gain setting in light drive circuitry 120. When light source 130 is configured to emit two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

In some embodiments, control circuitry 110 and light drive circuitry 120 may generate light drive parameters based on a metric. For example, back end processing 170 may receive information about received light signals, determine light drive parameters based on that information, and send corresponding information to control circuitry 110.

FIG. 2A shows an illustrative plot of a light drive signal including red light drive pulse 202 and IR light drive pulse 204 in accordance with some embodiments of the present disclosure. Light drive pulses 202 and 204 are illustrated as square waves. These pulses may include shaped waveforms rather than a square wave. The shape of the pulses may be generated by a digital signal generator, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a shaped pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130. Red light drive pulse 202 may have a higher amplitude than IR light drive pulse 204 since red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order to generate sensor signals that include physiological information that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulses. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark or returning to dark during that period. It will be understood that the particular square pulses illustrated in FIG. 2A are merely exemplary and that any suitable light drive scheme is possible. For example, light drive schemes may include shaped pulses, sinusoidal modulations, time division multiplexing other than as shown, frequency division multiplexing, phase division multiplexing, any other suitable light drive scheme, or any combination thereof.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current peak 226 may be generated in response to a light source being driven by red light drive pulse 202 of FIG. 2A, and peak 230 may be generated in response to a light source being driven by IR light drive pulse 204. Valleys 228 of detector current waveform 214 may be synchronous with periods of time during which no light is being emitted by the light source, or the light source is returning to dark, such as “off” periods 220. While no light is being emitted by a light source during the valleys, detector current waveform 214 may not fall all of the way to zero.

It will be understood that detector current waveform 214 may be an at least partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current as similar as possible to idealized detector current waveform 214.

Referring back to FIG. 1, front end processing circuitry 150, which may receive a one or more detection signals, such as detector current waveform 214, may include analog conditioning 152, analog-to-digital converter (ADC) 154, demultiplexer 156, digital conditioning 158, decimator/interpolator 160, and ambient subtractor 162.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some embodiments, one or more gain settings may be used in analog conditioning 152 to adjust the amplification of detector signal.

The conditioned analog signal may be processed by analog-to-digital converter 154, which may convert the conditioned analog signal into a digital signal. Analog-to-digital converter 154 may operate under the control of control circuitry 110. Analog-to-digital converter 154 may use timing control signals from control circuitry 110 to determine when to sample the analog signal. Analog-to-digital converter 154 may be any suitable type of analog-to-digital converter of sufficient resolution to enable a physiological monitor to accurately determine physiological parameters.

Demultiplexer 156 may operate on the analog or digital form of the detector signal to separate out different components of the signal. For example, detector current waveform 214 of FIG. 2B includes a red component corresponding to peak 226, an IR component corresponding to peak 230, and at least one ambient component corresponding to valleys 228. Demultiplexer 156 may operate on detector current waveform 214 of FIG. 2B to generate a red signal, an IR signal, a first ambient signal (e.g., corresponding to the ambient component corresponding to valley 228 that occurs immediately after the peak 226), and a second ambient signal (e.g., corresponding to the ambient component corresponding to valley 228 that occurs immediately after peak 230). Demultiplexer 156 may operate under the control of control circuitry 110. For example, demultiplexer 156 may use timing control signals from control circuitry 110 to identify and separate out the different components of the detector signal.

Digital conditioning 158 may perform any suitable digital conditioning of the detector signal. Digital conditioning 158 may include any type of digital filtering of the signal (e.g., low pass, high pass, band pass, notch, or any other suitable filtering), amplifying, performing an operation on the signal, performing any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal or signals.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 154. This may be achieved by applying one or more gains to the detection signal, by analog conditioning 152 to map the expected range of the signal to the full or close to full output range of analog-to-digital converter 154. The output value of analog-to-digital converter 154, as a function of the total analog gain applied to the detection signal, may be given as:

ADC Value=Total Analog Gain×[Ambient Light+LED Light]

Ideally, when ambient light is zero and when the light source is off, the analog-to-digital converter 154 will read just above the minimum input value. When the light source is on, the total analog gain may be set such that the output of analog-to-digital converter 154 may read close to the full scale of analog-to-digital converter 154 without saturating. This may allow the full dynamic range of analog-to-digital converter 154 to be used for representing the detection signal, thereby increasing the resolution of the converted signal. In some embodiments, the total analog gain may be reduced by a small amount so that small changes in the light level incident on the detector do not cause saturation of analog-to-digital converter 154.

However, if the contribution of ambient light is large relative to the contribution of light from a light source, the total analog gain applied to the detection current may need to be reduced to avoid saturating analog-to-digital converter 154. When the analog gain is reduced, the portion of the signal corresponding to the light source may map to a smaller number of analog-to-digital conversion bits. Thus, more ambient light noise in the input of analog-to-digital converter 154 may results in fewer bits of resolution for the portion of the signal from the light source. This may have a detrimental effect on the signal-to-noise ratio of the detection signal. Accordingly, passive or active filtering or signal modification techniques may be employed to reduce the effect of ambient light on the detection signal that is applied to analog-to-digital converter 154, and thereby reduce the contribution of the noise component to the converted digital signal.

Back end processing circuitry 170 may include processor 172 and memory 174. Processor 172 may be adapted to execute software, which may include an operating system and one or more applications, as part of performing the functions described herein. Processor 172 may receive and further process sensor signals received from front end processing circuitry 150. For example, processor 172 may determine one or more physiological parameters based on the received physiological signals. Processor 172 may include an assembly of analog or digital electronic components. Processor 172 may calculate physiological information. For example, processor 172 may compute one or more of fluid responsiveness, a blood oxygen saturation (e.g., arterial, venous, or both), pulse rate, respiration rate, respiration effort, blood pressure, hemoglobin concentration (e.g., oxygenated, deoxygenated, and/or total), any other suitable physiological parameters, or any combination thereof. Processor 172 may perform any suitable signal processing of a signal, such as any suitable scaling, band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from user interface 180. Additional input signals may be used by processor 172 in any of the calculations or operations it performs in accordance with back end processing circuitry 170 or monitor 104.

Memory 174 may include any suitable computer-readable media capable of storing information that can be interpreted by processor 172. In some embodiments, memory 174 may store calculated values, such as pulse rate, blood pressure, blood oxygen saturation, fiducial point locations or characteristics, initialization parameters, systemic vascular resistance, mean arterial pressure, cardiac output, central venous pressure, oxygen demand, adaptive filter parameters, fluid responsiveness parameters, any other calculated values, or any combination thereof, in a memory device for later retrieval. This information may be data or may take the form of computer-executable instructions, such as software applications, that cause a microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with user interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (e.g., an “SpO₂” or a regional oximetry measurement), fluid responsiveness information, pulse rate information, respiration rate and/or effort information, blood pressure information, hemoglobin concentration information, systemic vascular resistance, mean arterial pressure, cardiac output, central venous pressure, oxygen demand, any other parameters, and any combination thereof. Display 184 may include any type of display such as a cathode ray tube display, a flat panel display such as a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

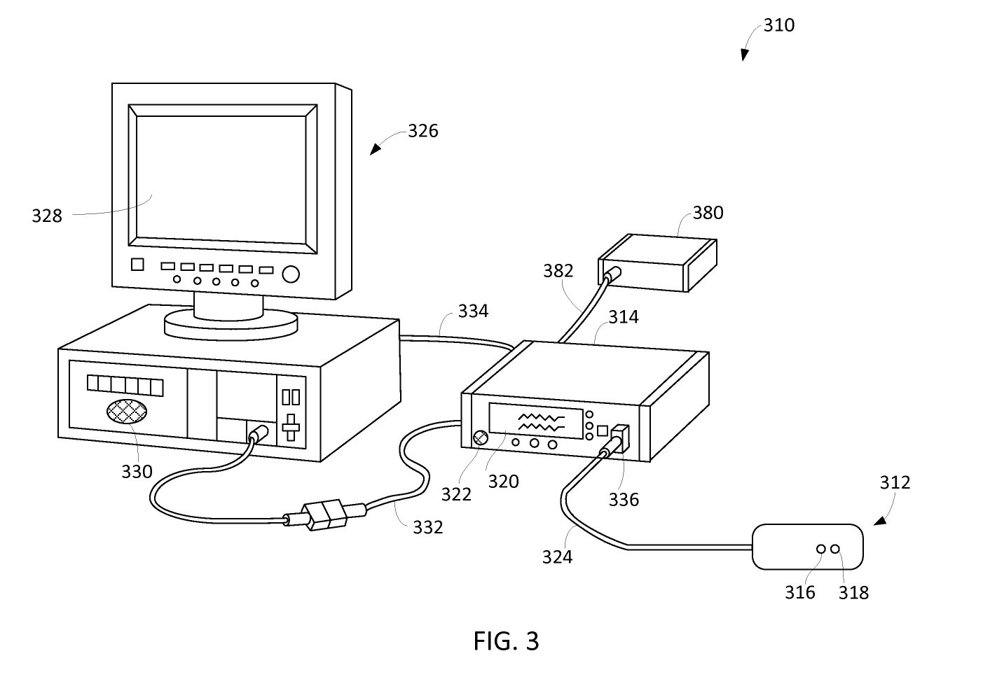

FIG. 3 is a perspective view of an illustrative physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body. In an example, an oximeter sensor may be located at a first position and a thermodilution sensor may be located at a second location. In another example, an oximeter sensor and a temperature sensor may be located near to one another or in the same structure.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected (not shown) to monitor 314. Monitor 314 may be configured to calculate physiological parameters based at least in part on data relating to light emission and light detection received from one or more sensor units such as sensor unit 312. For example, monitor 314 may be configured to determine fluid responsiveness, pulse rate, respiration rate, respiration effort, blood pressure, blood oxygen saturation (e.g., arterial, venous, regional, or a combination thereof), hemoglobin concentration (e.g., oxygenated, deoxygenated, and/or total), systemic vascular resistance, mean arterial pressure, cardiac output, central venous pressure, oxygen demand, any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324 at port 336. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's fluid responsiveness, blood oxygen saturation, and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, any of the processing components and/or circuits, or portions thereof, of FIGS. 1 and 3, including sensors 102 and 312 and monitors 104, 314, and 326, may be referred to collectively as processing equipment. For example, processing equipment may be configured to amplify, filter, sample and digitize an input signal from sensor 102 or 312 (e.g., using an analog-to-digital converter), and calculate physiological information from the digitized signal. Processing equipment may be configured to generate light drive signals, amplify, filter, sample and digitize detector signals, sample and digitize other analog signals, calculate physiological information from the digitized signal, perform any other suitable processing, or any combination thereof. In some embodiments, all or some of the components of the processing equipment may be referred to as a processing module.

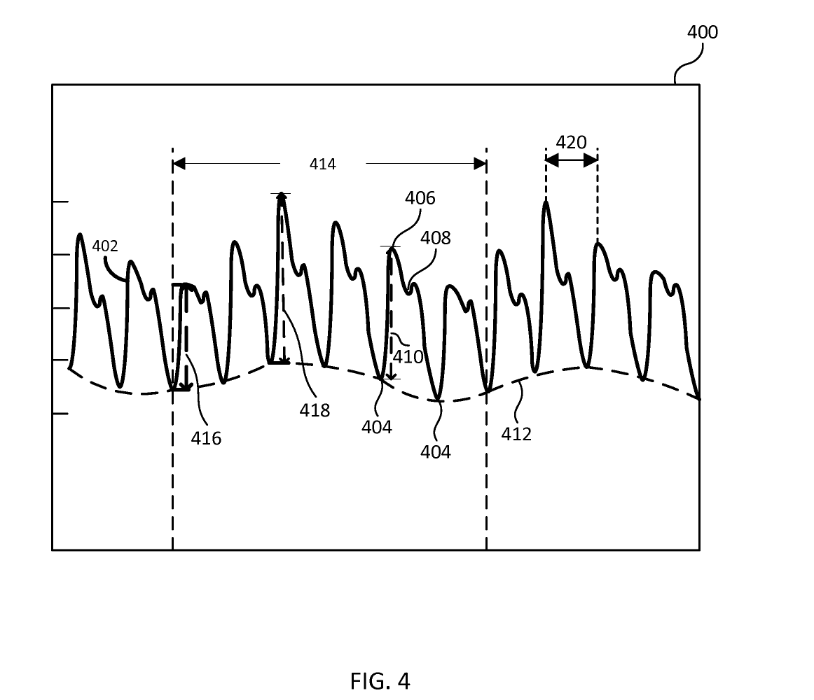

As described above, respiratory variation in the arterial blood pressure waveform is known to be a good predictor of a subject's fluid responsiveness. In particular, the PPV of a subject is known to be a good predictor of fluid responsiveness, but, as described above, requires invasive procedures to determine. Accordingly, determining respiratory variation in a PPG signal from a pulse oximeter may provide a non-invasive alternative to determining the PPV of a subject. Determination of fluid responsiveness in accordance with the present disclosure will be discussed with reference to FIGS. 4-11 below. Although a PPG signal from a pulse oximeter is used to illustrate embodiments of the present disclosure, it will be understood that the techniques described herein are not limited to PPG signals and pulse oximeters and may be applied to any suitable physiological signals and monitoring devices.

FIG. 4 shows an illustrative plot 400 of PPG waveform 402 reflecting respiratory modulations in accordance with some embodiments of the present disclosure. PPG waveform 402 may be generated, for example, by system 100 of FIG. 1 or system 310 of FIG. 3. As illustrated, PPG waveform 402 represents the absorption of light by a subject's tissue over time. PPG waveform 402 includes pulses where the absorption of light increases due to the increased volume of blood in the arterial blood vessel due to cardiac pulses. In some embodiments, pulses may be identified between adjacent valleys 404 and as illustrated may include a peak 406 and a dichrotic notch 408. The pulses include an upstroke between the first valley and the main peak. For example, an upstroke is depicted in FIG. 4 between the first valley 404 and peak 406. The amplitude of this upstroke is depicted as amplitude 410 measured from the first valley 404 to peak 406. Other amplitude values may be derived from the PPG waveform, such as a downstroke amplitude, average amplitude, or area under the pulse. In some embodiments, the amplitude of a pulse may be determined by subtracting a minimum value of PPG waveform 402 from a maximum value of PPG waveform 402 within a segment of PPG waveform 402 that generally corresponds to the period of a pulse. PPG waveform 402 also includes a varying baseline 412. PPG waveform 402 modulates above baseline 412 due to the pulses.

For most subjects, the PPG signal is affected by the subject's respiration, i.e. inhaling and exhaling, resulting in certain respiration modulations in the PPG waveform. FIG. 4 illustrates respiration modulations in PPG waveform 402 as a result of the subject's inhaling and exhaling. One type of respiratory modulation is the modulation of baseline 412 of PPG waveform 402. The effect of the subject's breathing in and out causes the baseline of the waveform 402 to move up and down, cyclically, with the subject's respiration. The baseline may be tracked by following any fiducial of PPG waveform 402, such as the peaks 406, valleys 404, dichrotic notches 408, median value, or any other fiducials. A second type of respiration-induced modulation of PPG waveform 402 is the modulation of pulse amplitudes. As the patient breathes in and out, the amplitudes of pulses decreases and increases, with larger amplitudes tending to coincide with the top of the baseline shift, and smaller amplitudes tending to coincide with the bottom of the baseline shift (though the larger and smaller amplitudes do not necessarily fall at the top and bottom of the baseline shift). A third respiratory type of modulation is the modulation of period 420 between pulses (also referred to as frequency modulation). Each of these modulations may be referred to as a respiratory component of PPG waveform 402, or a respiratory-induced modulation of PPG waveform 402. It should be noted that a particular individual may exhibit only the baseline modulation, or only the amplitude modulation, or only the frequency modulation, or any combination thereof. As referred to herein, a respiratory component of the PPG waveform 402 includes any one of these respiratory-induced modulations of PPG waveform 402, a measure of these modulations, or a combination of them.

The respiratory modulations of PPG waveform 402 can be affected by a subject's fluid status. For example, a hypovolemic subject may exhibit relatively larger respiratory variations of PPG waveform 402. When a subject loses fluid, the subject may have decreased cardiac output or stroke volume, which tends to increase the respiratory variations present in the subject's PPG waveform. Specifically, the baseline modulation, amplitude modulation, and frequency modulation may become more pronounced. Thus, larger respiratory modulations may indicate that the subject will respond favorably to fluid loading, whereas smaller respiratory modulations may indicate that a patient may not respond favorably to fluid loading. The respiratory modulations of the PPG waveform 402 may be identified and used to determine a subject's fluid responsiveness.

In some embodiments, a physiological monitor receives a PPG signal and determines a parameter indicative of fluid responsiveness based on the PPG signal. In some embodiments, the parameter indicative of fluid responsiveness is a measure of a subject's likely response to fluid therapy. In some embodiments, the parameter indicative of fluid responsiveness is a metric that reflects a degree of respiratory variation of the PPG signal. One example of a parameter indicative of fluid responsiveness is a measure of the amplitude modulations of the PPG signal, such as Delta POP (DPOP or ΔPOP, defined below). Another example of a parameter indicative of fluid responsiveness is a measure of the baseline modulation of the PPG. In some embodiments, other suitable metrics or combinations of metrics may be used to assess the respiratory modulation of the PPG signal. For example, a parameter indicative of fluid responsiveness may be based on the amplitudes or areas of acceptable pulses within a particular time frame or window. For example, as illustrated in FIG. 4, minimum amplitude 416 of the pulses within respiratory period 414 may be subtracted from maximum amplitude 418 within respiratory period 414 and then divided by an average or mean value of minimum amplitude 416 and maximum amplitude 418. In some embodiments, a parameter indicative of fluid responsiveness may be derived from the period or frequency of pulses within a time frame or window. For example, a modulation or variation in the period or frequency among two or more cardiac pulses may be used to derive a parameter indicative of fluid responsiveness. In general, the parameter indicative of fluid responsiveness may be based on one or more respiratory variations exhibited by the PPG waveform 402. Further, a parameter indicative of fluid responsiveness may be determined through the use of wavelet transforms, such as described in United States Patent Application Publication No. 2010/0324827, entitled “Fluid Responsiveness Measure,” which is hereby incorporated by reference in its entirety.

In some embodiments, DPOP is used as the parameter indicative of fluid responsiveness. The DPOP metric can be calculated from PPG waveform 402 for a particular time window as follows:

DPOP=(AMPₘₐₓ−AMPₘᵢₙ)/AMPₐᵥₑ (1)

where AMPₘₐₓ represents the maximum amplitude (such as maximum amplitude 418 in FIG. 4) during a time window (such as respiratory period 414 in FIG. 4), AMPₘᵢₙ represents the minimum amplitude (such as minimum amplitude 416 in FIG. 4) during the time window, and AMPₐᵥₑ is the average of the two, as follows:

AMPₐᵥₑ=(AMPₘₐₓ+AMPₘᵢₙ)/2 (2)

In some embodiments, AMPₘₐₓ and AMPₘᵢₙ may be measured at other locations of the PPG, such as within or along a pulse. DPOP is a measure of the respiratory variation in the AC portion of the PPG signal. DPOP is a unit-less value, and in some embodiments can be expressed as a percentage. In some embodiments, respiratory period 414 is one respiratory cycle (inhalation and exhalation). In some embodiments, respiratory period 414 is a fixed duration of time that approximates one respiratory cycle, such as 5 seconds, 10 seconds, or any other suitable duration. In some embodiments, respiratory period 414 may be adjusted dynamically based on the subject's calculated or measured respiration rate, so that the period is approximately the same as one respiratory cycle period. In some embodiments, a signal turning point detector may be used to identify the maximum and minimum points in the PPG signal, in order to calculate the upstroke amplitudes.

In some embodiments, it is desirable to determine the parameter indicative of fluid responsiveness by averaging the parameter as calculated in accordance with any of the embodiments described above over a second time window. For example, if DPOP is used as the parameter indicative of fluid responsiveness, and is calculated over a fixed duration of 10 seconds, it may be desirable to average the plurality of DPOP calculations performed over a fixed window of 120 seconds, effectively taking the average of 12 DPOP calculations to yield a parameter indicative of the subject's fluid responsiveness. Because of the desirability of obtaining an average of several instantaneous fluid responsiveness calculations, it is important that each of these instantaneous fluid responsiveness calculations be of good quality and accurate. Given the nature of the fluid responsiveness calculations described above, there are a number of occurrences within a PPG or other physiological signal used to determine fluid responsiveness that may interfere with the accuracy of instantaneous fluid responsiveness calculations, and in turn, with averages thereof. It is therefore desirable to detect these occurrences and adjust the determination of the subject's fluid responsiveness accordingly.

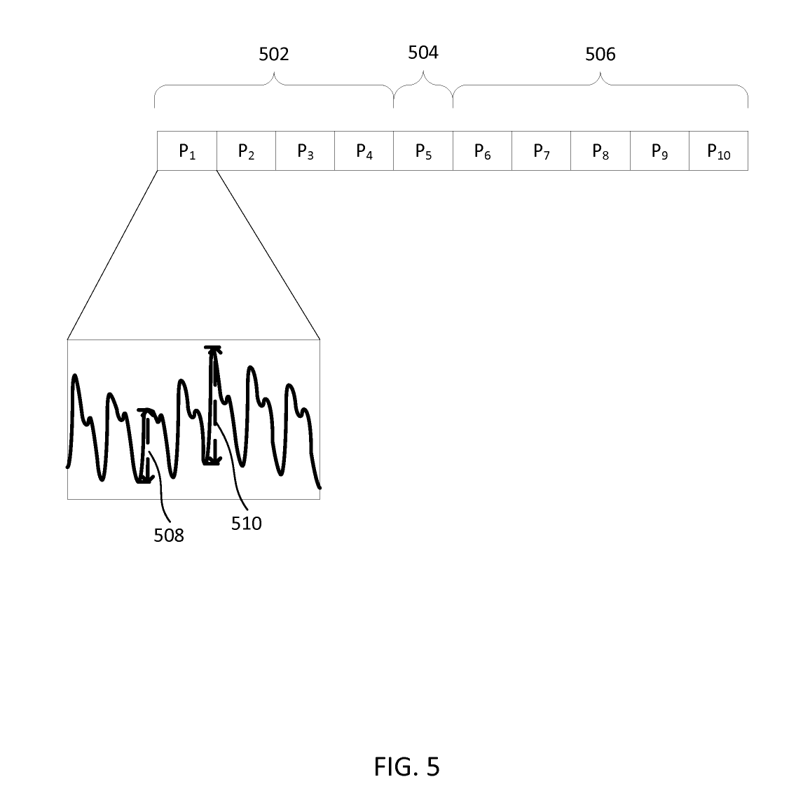

FIG. 5 shows an illustrative diagram of a PPG signal divided into segments P1-P10. FIG. 5 illustrates the need for identifying occurrences that interfere with the accuracy of instantaneous fluid responsiveness calculations in order to accurately determine fluid responsiveness of a subject over time. As described above, in some embodiments instantaneous fluid responsiveness calculations may be performed and an average of the instantaneous fluid responsiveness calculations may be taken. As illustrated in FIG. 5, the PPG signal may be divided into segments as represented by P1-P10. The period of each segment may generally correspond to the period of a respiratory cycle (e.g., 5 second, 10 seconds, or any other suitable period of time). Each segment may include respiratory modulations. For example, each segment may include pulse amplitudes reflecting respiration modulations (such as minimum amplitude 508 and maximum amplitude 510). In some embodiments, instantaneous fluid responsiveness calculations may be performed for each of segments P1-P10. The fluid responsiveness value that is outputted for display may be an average of the instantaneous fluid responsiveness calculations for segments P1-P10. However, if, for example, something occurs during segment P5 that causes rapid fluctuations or noise in the PPG signal that is not reflective of respiratory modulations, the instantaneous fluid responsiveness calculation for this segment may be inaccurate, which may cause an average of the instantaneous fluid responsiveness calculations for segments P1-P10 to be inaccurate. Accordingly, in accordance with some embodiments of the present disclosure, it may be desirable to detect such occurrences, and exclude the affected segments or the instantaneous fluid responsiveness calculations for the affected segments from the calculation of the fluid responsiveness parameter. For example, when rapid fluctuations or noise exists in segment P5, the fluid responsiveness parameter may be determined based on portions 502 and 506 of the PPG signal, and not based on portion 504 of the PPG signal. In other words, portion 504 (i.e., segment P5) is ignored.

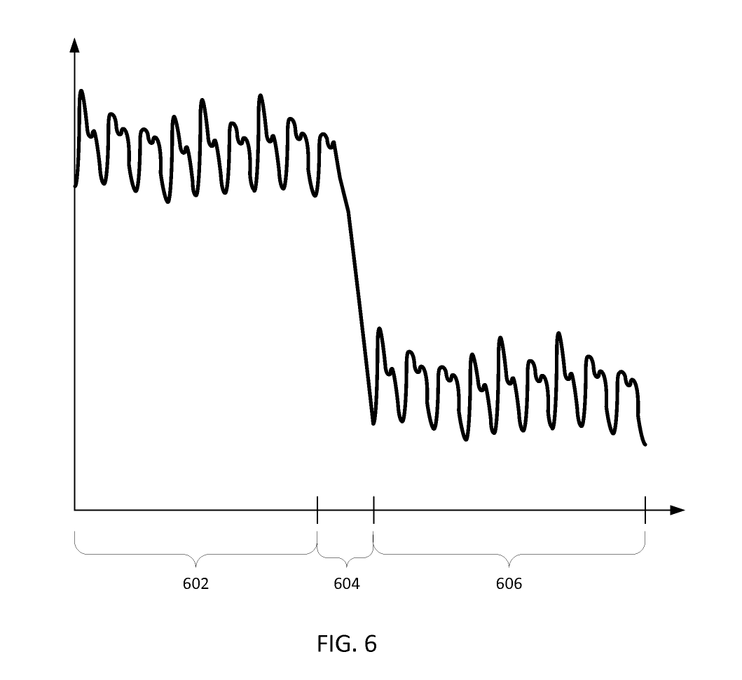

One type of occurrence in a PPG signal or other physiological signal used to determine fluid responsiveness that may interfere with the accuracy of instantaneous fluid responsiveness calculations is a gain change. As described above with respect to FIG. 1, sensor 102 of physiological monitoring system 100 may include light source 130 and detector 140, each of which may require gain adjustments from time to time during operation. It will be appreciated that in response to adjustments in gain, the output PPG signal may exhibit a dramatic change from one gain setting to another gain setting. FIG. 6 shows an illustrative plot of a PPG signal during a gain change. As can be seen in FIG. 6, there are three relevant time periods involved in the gain change. During time period 602, the PPG signal is being received at a first gain setting. Upon the adjustment of a gain (e.g., a light drive circuitry gain, an amplifier gain, or any other system gain) during time period 604, the PPG signal is in a transitory state until it settles down to a new level during time period 606. It will be appreciated by one of skill in the art that the PPG signal received during time period 604 may comprise both transient and noisy PPG components that would detrimentally affect calculations based on modulations of the PPG signal, such as determinations of fluid responsiveness during this time period. Accordingly, in some embodiments, it may be desirable to detect a gain change in a signal and determine fluid responsiveness based on the detected gain change.

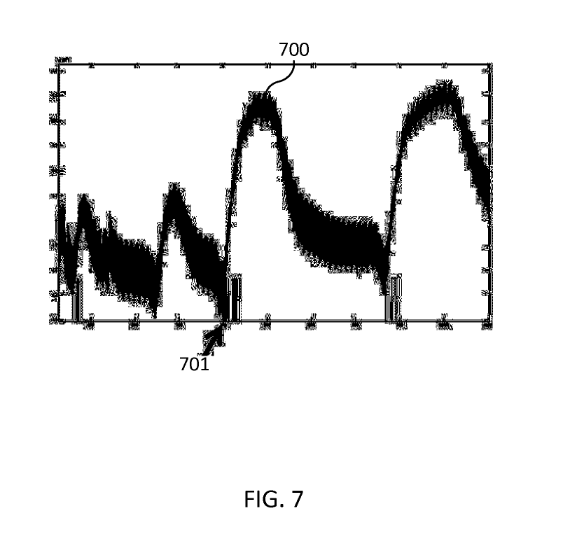

Another type of occurrence in a PPG signal or other physiological signal used to determine fluid responsiveness that may interfere with the accuracy of instantaneous fluid responsiveness calculations is the presence of excessive baseline modulations in the signal. FIG. 7 shows an illustrative plot of a PPG signal during excessive baseline modulations. Specifically, PPG signal 700 can be seen to have large baseline increases, as evident at point 701, for example. For similar reasons as described above with respect to gain changes, it may be desirable to refrain from calculating fluid responsiveness when such excessive baseline modulations occur in the PPG signal.

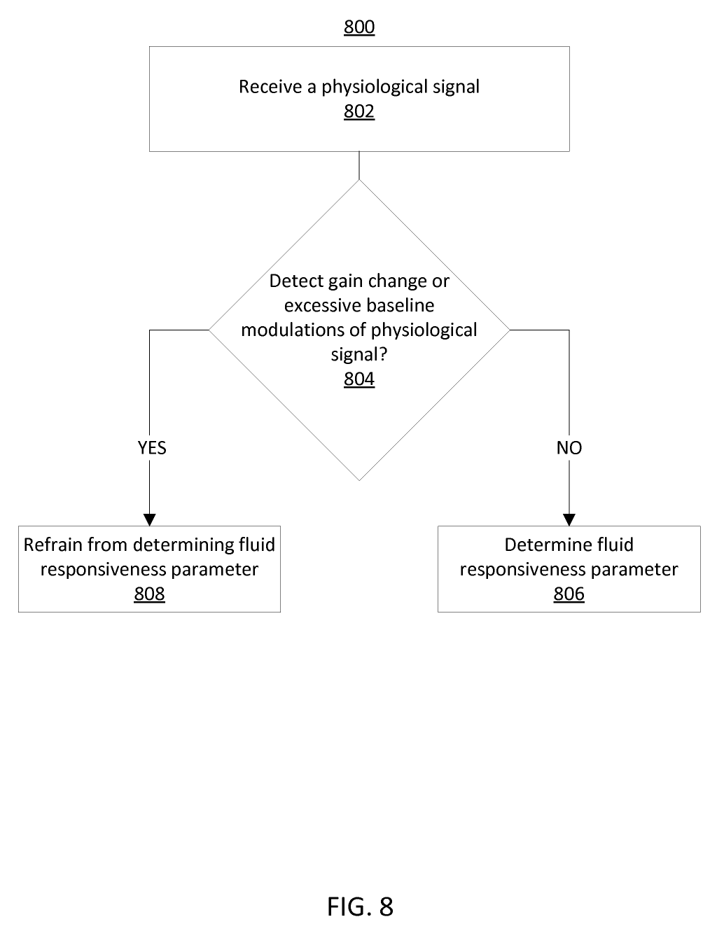

Determination of fluid responsiveness in the presence of gain changes and excessive baseline modulations in accordance with the present disclosure will be discussed with reference to FIGS. 8-11 below.

FIG. 8 shows illustrative steps 800 for determining fluid responsiveness in accordance with some embodiments of the present disclosure. Although exemplary steps are described herein, it will be understood that steps may be omitted and that any suitable additional steps may be added for determining respiration information. Although the steps described herein may be performed by any suitable device or system, in an exemplary embodiment, the steps may be performed by monitoring system 310, monitoring system 100, any components and modules thereof, and any combination thereof.

At step 802, the physiological monitoring system may receive a physiological signal. The physiological signal may be indicative of light attenuated by a subject. For example, the physiological signal may be a PPG signal received from a pulse oximeter.

At step 804, the physiological monitoring system may detect whether there is a gain change or whether there are excessive baseline modulations in the physiological signal. In some embodiments, at step 804, the gain change may be detected by analyzing the physiological signal or components thereof. For example, the gain change may be detected by analyzing a DC component associated with the signal, and determining if there is a rapid shift in the DC component that is indicative of a gain change. In other embodiments, the gain change may be detected by receiving an indication from the hardware of the physiological monitoring system that a gain setting was changed. For example, an indication may be generated when the gain applied to light source 130 or the gain applied to a detector signal is adjusted by the physiological monitoring system.

In some embodiments, at step 804, excessive baseline modulations may be detected by band pass filtering a PPG signal around an expected range of respiration (which may be fixed or based on a respiration rate determined by a suitable algorithm) to extract a baseline signal. The baseline signal may then be broken into 5 second windows, for example. The signal may then be normalized by dividing by average pulse amplitude within each window. The resulting signal may then be compared with a threshold value and any windows that contain values that exceed the threshold may be flagged as potentially having large baseline shifts. In some embodiments, the variation in baseline may be compared to a standard deviation based threshold for a longer window.

If no gain change is detected, and no excessive baseline modulations are detected at step 804, the physiological monitoring system may proceed to step 806 and determine the fluid responsiveness parameter. The fluid responsiveness parameter may be determined in accordance with any of the above-mentioned methods. For example, the fluid responsiveness parameter may be determined by identifying maximum and minimum amplitudes during a time window and dividing a difference between the amplitudes by an average of the amplitudes.

If a gain change and/or excessive baseline modulations are detected at step 804, the physiological monitoring system may proceed to step 808 and refrain from determining the fluid responsiveness parameter. In some embodiments, the physiological monitoring system may refrain from determining an instantaneous fluid responsiveness parameter until the gain change is complete and the adjusted gain is achieved and/or the excessive baseline modulations are no longer detected. In some embodiments, the physiological monitoring system may refrain from using or averaging instantaneous fluid responsiveness parameter values determined during a gain change and excessive baseline modulations. For example, during a gain change and/or when baseline modulations exceed a predetermined threshold, the system may flag any instantaneous fluid responsiveness parameter values determined during this time, and ignore them from a calculation of an average fluid responsiveness parameter.

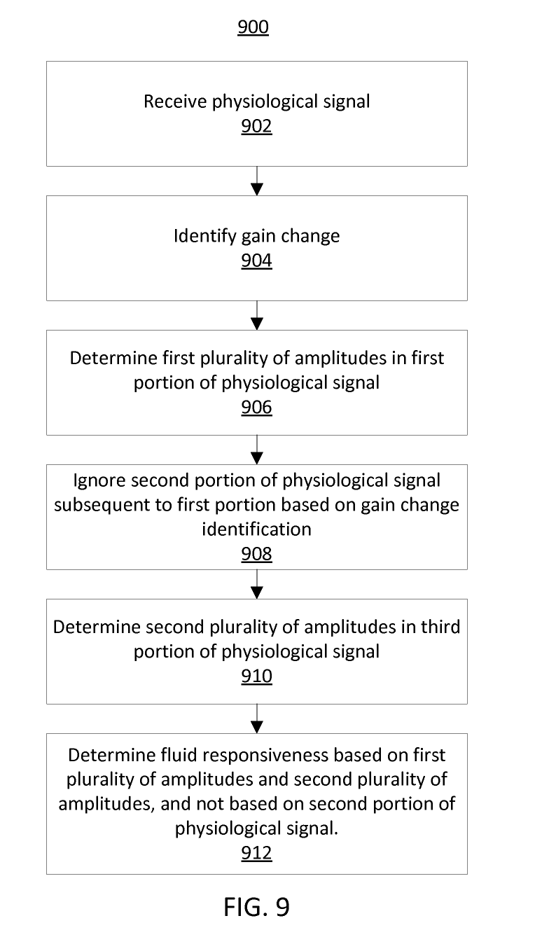

FIG. 9 shows illustrative steps 900 for determining fluid responsiveness in accordance with some embodiments of the present disclosure. Although exemplary steps are described herein, it will be understood that steps may be omitted and that any suitable additional steps may be added for determining respiration information. Although the steps described herein may be performed by any suitable device or system, in an exemplary embodiment, the steps may be performed by monitoring system 310, monitoring system 100, any components thereof, and any combination thereof.

At step 902, the system may receive a physiological signal. As described above with respect to step 702 of FIG. 7, the physiological signal may be indicative of light attenuated by a subject. For example, the physiological signal may be a PPG signal received from a pulse oximeter.

At step 904, the system may identify a gain change. In some embodiments, the gain change may be identified by analyzing the physiological signal or by receiving an indication from the hardware of the physiological monitoring system that a gain setting was changed, as described above with respect to step 804 of FIG. 8. In some embodiments, the gain change indication may be received from a subcomponent of the physiological monitoring system, another system external to the physiological monitoring system, or any suitable combination thereof.

At step 906, the system may determine a first plurality of amplitudes in a first portion of the physiological signal. In some embodiments, the system may determine one or more maximum amplitudes and minimum amplitudes in the first portion of the physiological signal. In some embodiments, the system may determine maximum amplitudes and minimum amplitudes for each of several segments of the first portion of the physiological signal. In some embodiments, the duration of each segment of the first portion of the physiological signal may correspond to the duration of a respiratory cycle of the subject. For example, the duration of each segment may depend on the respiration rate of the subject as determined by a pulse oximeter or other suitable means. In some embodiments, the duration of each segment of the first portion of the physiological signal may be a fixed duration. For example, the duration of each segment may be approximately ten seconds. In some embodiments, the system may determine instantaneous values indicative of fluid responsiveness for each of the segments of the first portion of the physiological signal. For example, the system may determine DPOP according to Eqs. 1 and 2 above for each segment in the first portion of the physiological signal based on maximum and minimum amplitudes determined for each segment. In some embodiments, the first portion of the physiological signal may correspond to portion 502 of FIG. 5.

At step 908, the system may ignore a second portion of the physiological signal subsequent to the first portion based on the gain change identified at step 904. In some embodiments, the system may refrain from determining amplitudes or instantaneous values indicative of fluid responsiveness during the second portion of the physiological signal if a gain change is detected during the second portion. In some embodiments, the system may ignore the second portion of the physiological signal in the determination of a fluid responsiveness parameter. In some embodiments, the second portion of the physiological signal may correspond to portion 504 of FIG. 5.

At step 910, the system may determine a second plurality of amplitudes in a third portion of the physiological signal subsequent to the second portion of the physiological signal. The second plurality of amplitudes may be determined in the same way as the first plurality of amplitudes as described above with respect to step 906. Similarly, the system may determine amplitudes for each of several segments of the third portion of the physiological signal in the same way as described above with respect to step 906, with the duration of each segment determined in the same way as described therein. Furthermore, the system may determine instantaneous values indicative of fluid responsiveness for each of the segments of the third portion of the physiological signal in the same way as described above with respect to step 906. In some embodiments, the third portion of the physiological signal may correspond to portion 506 of FIG. 5.

At step 912, the system may determine fluid responsiveness of the subject based on the first plurality of amplitudes and the second plurality of amplitudes, and not based on the ignored second portion of the physiological signal. In some embodiments, the system may determine the fluid responsiveness of the subject by combining the instantaneous values indicative of fluid responsiveness for each of the segments of the first portion of the physiological signal and the third portion of the physiological signal in any suitable way. For example, the system may determine the fluid responsiveness of the subject by calculating an average of the instantaneous values indicative of fluid responsiveness determined from each of the segments of the first portion of the physiological signal (e.g., portion 502 of FIG. 5) and the third portion of the physiological signal (e.g., portion 506 of FIG. 5). In some embodiments, outlier values may be removed before averaging.

Although embodiments of FIG. 9 have been described above with respect to determining fluid responsiveness in the presence of gain changes, it will be appreciated that the steps of FIG. 9 are equally applicable to determining fluid responsiveness in the presence of excessive baseline modulations. Accordingly, in some embodiments, step 904 may be replaced with a step for identifying information indicative of excessive baseline modulations. Information indicative of excessive baseline modulations may be detected by extracting a baseline signal, dividing the baseline signal into windows, normalizing the signal, and comparing it to a threshold value as described with respect to step 804 of FIG. 8. Steps 906 through 912 may then be performed as described above, except that the second portion may be ignored (in step 908) and not used to determine fluid responsiveness (in step 912) based on information indicative of excessive baseline modulations, as opposed to a gain change identification.

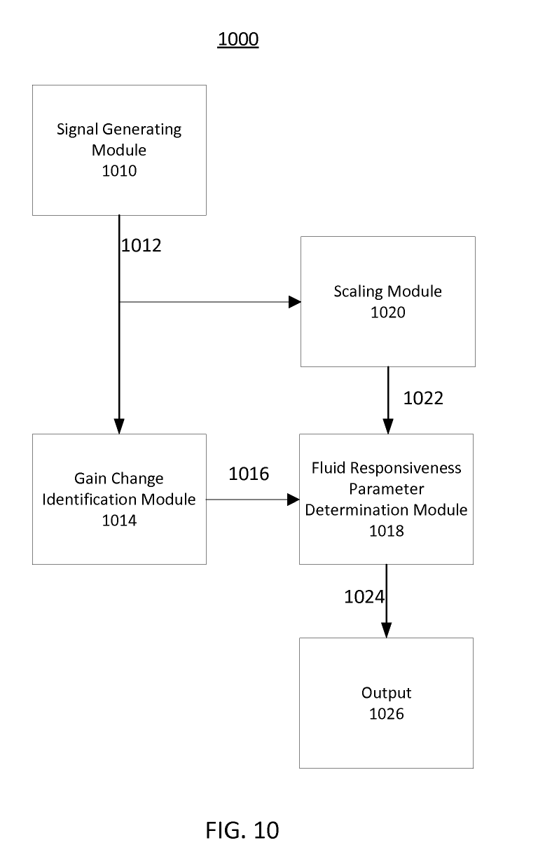

An illustrative physiological monitor 1000 for monitoring fluid responsiveness of a subject is shown in FIG. 10. The monitor 1000 includes a signal generating module 1010. In some embodiments, signal generating module 1010 may include any suitable combination of components of monitor 100 as described with respect to FIG. 1 for generating a physiological signal. For example, signal generating module 1010 may include light drive circuitry 120, control circuitry 110, and front end processing circuitry 150 as described above with respect to FIG. 1, and may be configured to generate signals and process them as described above. In some embodiments, signal generating module 1010 may include fewer components or additional components (e.g., sensor 102). Signal generating module 1010 includes one or more adjustable gains and generates a physiological signal. In some embodiments, the physiological signal may be a signal indicative of light attenuated by a subject. For example, the physiological signal may be a PPG signal generated by a pulse oximeter as described above with respect to FIGS. 1-3. Signal generating module 1010 may optionally generate a gain change indication, which indicates if a gain associated with signal generating module 1010 has been adjusted. For example, if a light drive circuitry gain, amplifier gain, or any other gain of signal generating module 1010 is adjusted during operation, signal generating module 1010 may generate an indication thereof (e.g., by setting a gain change flag).

Signal generating module 1010 generates output 1012. Output 1012 may include the physiological signal and the gain change indication. In some embodiments, output 1012 is passed to gain change identification module 1014. Gain change identification module 1014 may be configured to identify a gain change in the signal generating module. Gain change identification module 1014 may identify a gain change by analyzing the physiological signal or by receiving an indication from the hardware of the physiological monitoring system that a gain setting was changed as described above with respect to step 804 of FIG. 8. In some embodiments, gain change identification module 1014 may include any suitable combination of components of monitor 100 as described with respect to FIG. 1 for analyzing and processing a physiological signal. For example, gain change identification module 1014 may include front end processing circuitry 150, back end processing circuitry 170, any components thereof, and/or any suitable combination thereof as described above with respect to FIG. 1, and may be configured to receive signals and process them as described above. In some embodiments, gain change identification module 1014 may include fewer components or additional components. Gain change identification module 1014 generates output 1016 that is passed to fluid responsiveness parameter determination module 1018. Output 1016 may include a gain change identification.

In some embodiments, output 1012 of signal generating module 1010 may also be passed to scaling module 1020. Scaling module 1020 may be configured to remove the effect of any gains applied by signal generating module 1010 and generate an output 1022 that includes the scaled physiological signal, which is passed to fluid responsiveness parameter determination module 1018. This scaling may compensate for nonlinear effects of a gain change, which may cause differences in fluid responsiveness calculations determined before and after gain changes. Thus, the scaling may ensure that similar fluid responsiveness parameter determinations would be obtained for different gain settings. In some embodiments, scaling module 1020 may include any suitable combination of components of monitor 100 as described with respect to FIG. 1 for analyzing and processing a physiological signal. For example, scaling module 1020 may include front end processing circuitry 150, back end processing circuitry 170, any components thereof, and/or any suitable combination thereof as described above with respect to FIG. 1, and may be configured to receive signals and process them as described above. In some embodiments, scaling module 1020 may include fewer components or additional components. It will be understood that scaling module 1020 is optional and in some embodiments, scaling module 1020 is not used and output 1012 of signal generating module 1010 may be passed directly to fluid responsiveness parameter determination module 1018.

In some embodiments, fluid responsiveness parameter determination module 1018 may be configured to determine fluid responsiveness in a subject in accordance with any of the techniques described in the present disclosure. For example, fluid responsiveness parameter determination module 1018 may repeatedly calculate instantaneous fluid responsiveness values based on amplitudes determined within each 10 second instantaneous window, and if no gain change is identified in the 120 second calculation window, may calculate a fluid responsiveness parameter based on each of the twelve instantaneous values over the calculation window. For example, fluid responsiveness parameter determination module 1018 may take an average of all twelve of the instantaneous values determined over the 120 second calculation window.

In some embodiments, when a gain change indication is received from gain change identification module 1014, fluid responsiveness parameter determination module 1018 may refrain from calculating instantaneous fluid responsiveness values during the identified gain change or exclude any calculated instantaneous fluid responsiveness values from the calculation of the fluid responsiveness parameter. For example, if one of the twelve 10 second windows in a 120 second calculation window corresponds to a gain change, the fluid responsiveness parameter determination module 1018 may calculate a fluid responsiveness parameter based only on the other eleven instantaneous values over the calculation window. In some embodiments, the fluid responsiveness parameter determination module 1018 may wait to calculate the fluid responsiveness parameter until it receives twelve 10 second windows free from any gain change identification. In some embodiments, the fluid responsiveness parameter determination module 1018 may use the most recent twelve 10 second instantaneous fluid responsiveness values free from any gain change identification in its determination of the fluid responsiveness parameter, skipping any intermittent windows corresponding to identified gain changes. It will be understood that the foregoing examples are merely illustrative and that windows of any suitable size and of any suitable number may be used to determine the fluid responsiveness parameter.

In some embodiments, fluid responsiveness parameter determination module 1018 may adjust the parameter according to a percent modulation of the physiological signal. For example, the fluid responsiveness parameter determination module may determine an amplitude component of the physiological signal and divide the amplitude component by a baseline component of the physiological signal to obtain a percent modulation of the physiological signal. Fluid responsiveness parameter determination module 1018 may then correct or normalize the previously determined fluid responsiveness parameter based on the percent modulation of the physiological signal.

In some embodiments, when a gain change is identified in the physiological signal, fluid responsiveness parameter determination module 1018 may account for nonlinear effects of the gain change by applying a scaling factor to instantaneous fluid responsiveness values calculated before and after the identified gain change, and then determining the fluid responsiveness parameter based on the scaled instantaneous values.

In some embodiments, fluid responsiveness parameter determination module 1018 may determine parameter indicative of fluid responsiveness 1024 in accordance with any of the above-mentioned techniques, including those discussed above with respect to FIGS. 8 and 9, and pass it to output 1026. In some embodiments, fluid responsiveness parameter determination module 1018 may include any suitable combination of components of monitor 100 as described with respect to FIG. 1 for analyzing and processing a physiological signal. For example, fluid responsiveness parameter determination module 1018 may include front end processing circuitry 150, back end processing circuitry 170, any components thereof, and/or any suitable combination thereof as described above with respect to FIG. 1, and may be configured to receive signals and process them as described above. In some embodiments, fluid responsiveness parameter determination module 1018 may include fewer components or additional components.

Output 1026 may include display 184 and/or communication interface 190 of monitor 104 as described above with respect to FIG. 1, displays 320 and/or 328 of physiological monitoring system 310 as described above with respect to FIG. 3, any other suitable output, or any other suitable combination thereof. For example, the parameter indicative of fluid responsiveness may be output to be displayed on display 320, display 328, display 184, or may be output to another device via communication interface 190, so that a clinician may diagnose a subject's condition and provide treatment in response thereto.

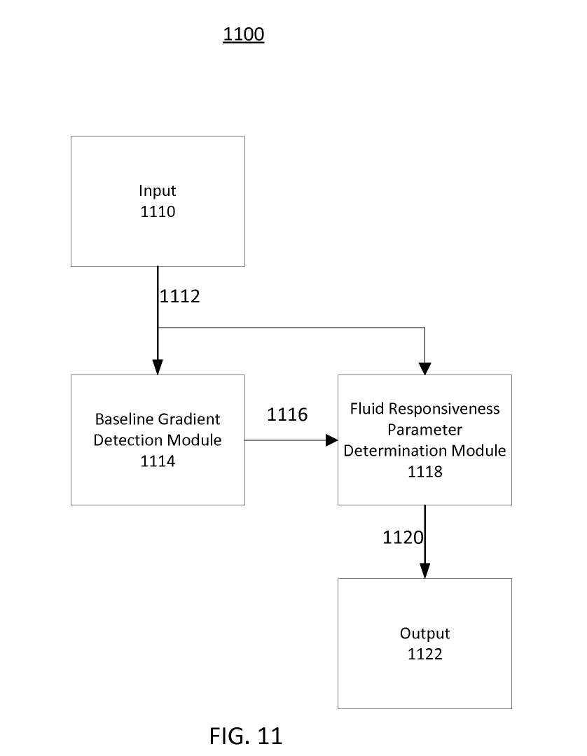

An illustrative physiological monitor 1100 for monitoring fluid responsiveness of a subject is shown in FIG. 11. Monitor 1100 includes an input 1110. In some embodiments, input 1110 may include any suitable combination of components of monitor 100 for receiving a signal as described with respect to FIG. 1. For example, input 1110 may include sensor 102, light drive circuitry 120, control circuitry 110, and front end processing circuitry 150 as described above with respect to FIG. 1, and may be configured to receive, generate and process signals as described above. In some embodiments, input 1110 may include fewer components or additional components. Input 1110 receives a physiological signal. In some embodiments, the physiological signal may be a signal indicative of light attenuated by a subject. For example, the physiological signal may be a PPG signal generated by a pulse oximeter as described above with respect to FIGS. 1-3.

Input generates output 1112. Output 1112 may include the physiological signal. In some embodiments, output 1112 is passed to baseline gradient detection module 1114. Baseline gradient detection module 1114 may be configured to detect excessive baseline modulations in output 1112 as described above with respect to step 804 of FIG. 8. For example, excessive baseline modulations may be detected by extracting a baseline signal, dividing the baseline signal into windows, normalizing the signal, and comparing it to a threshold value. In some embodiments, baseline gradient detection module 1114 may include any suitable combination of components of monitor 100 as described with respect to FIG. 1 for analyzing and processing a physiological signal. For example, baseline gradient detection module 1114 may include front end processing circuitry 150, back end processing circuitry 170, any components thereof, and/or any suitable combination thereof as described above with respect to FIG. 1, and may be configured to receive signals and process them as described above. In some embodiments, baseline gradient detection module 1114 may include fewer components or additional components. Baseline gradient detection module 1114 generates output 1116 that is passed to fluid responsiveness parameter determination module 1118. Output 1116 may include information indicative of excessive baseline modulations. In some embodiments, output 1112 may also be passed to fluid responsiveness parameter determination module 1118.

In some embodiments, fluid responsiveness parameter determination module 1118 may be configured to determine fluid responsiveness in a subject in accordance with any of the techniques described in the present disclosure. For example, fluid responsiveness parameter determination module 1118 may repeatedly calculate instantaneous fluid responsiveness values based on amplitudes determined within each 10 second instantaneous window, and if excessive baseline modulations are not identified in the 120 second calculation window, may calculate a fluid responsiveness parameter based on each of the twelve instantaneous values over the calculation window. For example, fluid responsiveness parameter determination module 1018 may take an average of all twelve of the instantaneous values determined over the 120 second calculation window.

In some embodiments, when information indicative of excessive baseline modulations is received from baseline gradient detection module 1114, fluid responsiveness parameter determination module 1118 may refrain from calculating instantaneous fluid responsiveness values during the excessive baseline modulations or exclude any calculated instantaneous fluid responsiveness values from the calculation of the fluid responsiveness parameter. For example, if one of the twelve 10 second windows in a 120 second calculation window corresponds to excessive baseline modulations, the fluid responsiveness parameter determination module 1118 may calculate a fluid responsiveness parameter based only on the other eleven instantaneous values over the calculation window. In some embodiments, the fluid responsiveness parameter determination module 1118 may wait to calculate the fluid responsiveness parameter until it receives twelve 10 second windows free from excessive baseline modulations. In some embodiments, the fluid responsiveness parameter determination module 1118 may use the most recent twelve 10 second instantaneous fluid responsiveness values free from excessive baseline modulations in its determination of the fluid responsiveness parameter, skipping any intermittent windows corresponding to excessive baseline modulations. It will be understood that the foregoing examples are merely illustrative and that windows of any suitable size and of any suitable number may be used to determine the fluid responsiveness parameter.

In some embodiments, fluid responsiveness parameter determination module 1118 may adjust the parameter according to a percent modulation of the physiological signal. For example, the fluid responsiveness parameter determination module may determine an amplitude component of the physiological signal and divide the amplitude component by a baseline component of the physiological signal to obtain a percent modulation of the physiological signal. Fluid responsiveness parameter determination module may then correct or normalize the previously determined fluid responsiveness parameter based on the percent modulation of the physiological signal.

In some embodiments, fluid responsiveness parameter determination module 1118 may determine a parameter indicative of fluid responsiveness 1120 in accordance with any of the above-mentioned techniques, including those discussed above with respect to FIGS. 8 and 9, and pass it to output 1122. In some embodiments, fluid responsiveness parameter determination module 1118 may include any suitable combination of components of monitor 100 as described with respect to FIG. 1 for analyzing and processing a physiological signal. For example, fluid responsiveness parameter determination module 1118 may include front end processing circuitry 150, back end processing circuitry 170, any components thereof, and/or any suitable combination thereof as described above with respect to FIG. 1, and may be configured to receive signals and process them as described above. In some embodiments, fluid responsiveness parameter determination module 1118 may include fewer components or additional components.

Output 1122 may include display 184 and/or communication interface 190 of monitor 104 as described above with respect to FIG. 1, displays 320 and/or 328 of physiological monitoring system 310 as described above with respect to FIG. 3, any other suitable output, or any other suitable combination thereof. For example, the parameter indicative of fluid responsiveness may be output to be displayed on display 320, display 328, display 184, or may be output to another device via communication interface 190, so that a clinician may diagnose a subject's condition and provide treatment in response thereto.

It will be understood that while FIGS. 10 and 11 show separate systems for determining fluid responsiveness in the presence of gain changes and baseline changes, a system may be provided in accordance with the present disclosure that includes both gain change identification module 1014 of FIG. 10 and baseline gradient detection module 1114 of FIG. 11 so that fluid responsiveness can be accurately determined in the presence of both gain changes and baseline changes.

It will also be understood that while various embodiments of the present disclosure refer to determining amplitudes in first and third portions of a physiological signal when a gain change or excessive baseline modulations occur in an intermediary second portion, fluid responsiveness can be determined without determining such amplitudes. For example, in some embodiments, fluid responsiveness can be determined based on the first and third portions using other calculation techniques (e.g., frequency domain techniques) that can represent the amplitude of pulses. In addition, in some embodiments fluid responsiveness can be determined based on other types of respiratory modulations of a physiological signal (e.g., baseline respiratory modulations and/or frequency respiratory modulations).

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A physiological monitor for monitoring fluid responsiveness of a subject comprising:
a signal generating module configured to generate a physiological signal that is indicative of light attenuated by a subject;
a gain change identification module configured to identify a gain change in the signal generating module; and
a fluid responsiveness parameter determination module configured to:
receive the physiological signal from the signal generating module;
receive the gain change identification from the gain change identification module;
determine a first plurality of amplitudes in a first portion of the physiological signal;
ignore a second portion of the physiological signal, subsequent to the first portion, based on the received gain change identification;
determine a second plurality of amplitudes in a third portion of the physiological signal, subsequent to the second portion; and
determine the parameter indicative of fluid responsiveness based on the first plurality of amplitudes and the second plurality of amplitudes, and not based on the ignored second portion of the physiological signal.

2. The monitor of claim 1, wherein the first portion of the physiological signal comprises a first plurality of segments, wherein the third portion of the physiological signal comprises a second plurality of segments, and wherein the fluid responsiveness parameter determination module is configured to:
determine a first plurality of instantaneous values indicative of fluid responsiveness for the plurality of first segments;
determine a second plurality of instantaneous values indicative of fluid responsiveness for the plurality of second segments; and
determine the parameter indicative of fluid responsiveness by combining the first plurality of instantaneous values and the second plurality of instantaneous values.

3. The monitor of claim 2, wherein combining the first plurality of instantaneous values and the second plurality of instantaneous values comprises averaging the first plurality of instantaneous values and the second plurality of instantaneous values.

4. The monitor of claim 2, wherein the duration of each of the first plurality of segments and the second plurality of segments corresponds to at least the duration of a respiratory cycle of the subject.

5. The monitor of claim 2, wherein the duration of each of the first plurality of segments and the second plurality of segments is approximately ten seconds.

6. The monitor of claim 2, wherein the first, second, and third portions of the physiological signal together comprise a predetermined period of time over which the parameter indicative of fluid responsiveness is determined, wherein the fluid responsiveness parameter determination module is configured to combine a predetermined number of instantaneous values in the predetermined period of time to determine the parameter indicative of fluid responsiveness when a gain change is not identified during the predetermined period of time, and wherein the fluid responsiveness parameter determination module is configured to combine less than the predetermined number of instantaneous values to determine the parameter indicative of fluid responsiveness when a gain change is identified during the predetermined period of time.

7. The monitor of claim 1, wherein the fluid responsiveness parameter determination module is configured to determine the parameter indicative of fluid responsiveness by dividing a difference between a maximum amplitude and a minimum amplitude by an average of the maximum and minimum amplitudes.

8. The monitor of claim 1, wherein the gain change identification module is configured to identify a gain change based on at least one of a detected shift in the physiological signal and an indication that a gain setting was changed in the signal generating module.

9. The monitor of claim 1, wherein the gain change comprises at least one of a gain change applied to a light emitter and a gain change applied to a detector signal.

10. The monitor of claim 1, further comprising a scaling module configured to scale the physiological signal to remove the effect of the gains applied by the signal generating module to generate a scaled physiological signal, wherein the physiological signal received at the fluid responsiveness parameter determination module comprises the scaled physiological signal.

11. The monitor of claim 1, wherein the fluid responsiveness parameter determination module is configured to determine the parameter indicative of fluid responsiveness by:
dividing an amplitude component of the physiological signal by a baseline component of the physiological signal to generate a percent modulation of the physiological signal; and
adjusting the parameter indicative of fluid responsiveness based on the percent modulation of the physiological signal.

12. The monitor of claim 1, wherein the fluid responsiveness parameter determination module is configured to apply nonlinear scaling in the determination of the parameter indicative of fluid responsiveness to compensate for nonlinear effects of a gain change.

13. The monitor of claim 1, wherein the signal generating module comprises:
a light source;
light drive circuitry comprising a first adjustable gain;
a detector; and
an amplifier comprising a second adjustable gain.

14. The monitor of claim 1, wherein the physiological signal comprises a photoplethysmogram signal.

15. The monitor of claim 1, wherein the fluid responsiveness parameter determination module is configured to determine a third plurality of amplitudes in the second portion of the physiological signal, and wherein the fluid responsiveness parameter determination module is configured to determine the parameter indicative of fluid responsiveness not based on the third plurality of amplitudes.

16. A physiological monitor for monitoring fluid responsiveness of a subject, comprising:
an input configured to receive a physiological signal;
a baseline gradient detection module configured to detect excessive baseline modulations of the physiological signal that exceed a predetermined threshold; and
a fluid responsiveness parameter determination module configured to:
receive the physiological signal;
receive information indicative of excessive baseline modulations from the baseline gradient detection module;
determine a fluid responsiveness parameter based on the physiological signal; and
refrain from determining the fluid responsiveness parameter based on the information indicative of excessive baseline modulations.

17. The monitor of claim 16, wherein the baseline gradient detection module is further configured to:
extract a baseline component from the physiological signal;
divide the baseline component into a plurality of fixed time intervals;
normalize the baseline component by dividing the baseline component by an average amplitude associated with each time interval; and
compare the normalized baseline component to the predetermined threshold to detect excessive baseline modulations of the physiological signal.

18. The monitor of claim 16, wherein the predetermined threshold is based on a standard deviation associated with the physiological signal.

19. The monitor of claim 16, wherein the physiological signal comprises a first portion comprising a first plurality of segments, a second portion subsequent to the first portion, and a third portion subsequent to the second portion comprising a second plurality of segments, and wherein the fluid responsiveness parameter determination module is further configured to:
determine a first plurality of instantaneous values indicative of fluid responsiveness for the plurality of first segments;
ignore the second portion based on the received information indicative of excessive baseline modulations;
determine a second plurality of instantaneous values indicative of fluid responsiveness for the plurality of second segments; and
determine the fluid responsiveness parameter by combining the first plurality of instantaneous values and the second plurality of instantaneous values.

20. The monitor of claim 19, wherein combining the first plurality of instantaneous values and the second plurality of instantaneous values comprises averaging the first plurality of instantaneous values and the second plurality of instantaneous values.
